(19) United States
(12) Patent Application Publication
(10) Pub. No.: US 2003/0162694 A1
(43) Pub. Date: Aug. 28, 2003

(54) Novel treatment for pathological aggression

(76) Inventors: James Meyerhoff, Silver Spring, MD (US); Lucille A. Lumley, Silver Spring, MD (US)

Correspondence Address: James L. Meyerhoff, 2936 Woodstock Ave, Silver Spring, MD, 20910, US

(21) Appl. No.: 10/357,431

(22) Filed: Feb. 4, 2003

Related U.S. Application Data
(60) Provisional application No. 60/353,437, filed on Feb. 4, 2002.

(51) Int. Cl. A61K 38/16; A61K 31/66

(52) U.S. Cl. 514/7; 514/19; 514/120

(57) ABSTRACT

Compounds of the general class of substituted pentanedioic acids, where the substituted moiety might be (subclass 1) a sulfanyl alkyl group; (subclass 2) an halogenated benzyl and a phosphinyl group; (subclass 3) a phosphonomethyl group, may be used for prevention, management and/or treatment of hyperaggressive behavior arising from environmental or social conditions, injury or disease, as may [2-(pentafluorophenylmethyl) hydroxyphosphinyl]methyl-pentanedioic acid, and/or other related substituted pentanedioic acids that inhibit NAALADase or mimic NAAG, and/or 2-(phosphonomethyl)-pentanedioic acid (2-PMPA) and/or beta-N-acetyl-aspartyl-glutamate (NAAG) and/or alpha N-acetyl-aspartyl-glutamate.

This application claims benefit to provisional application Ser. No. 60/353,437 filed Feb. 4, 2002.

### FIELD OF THE INVENTION

This invention relates to the use of compositions containing containing compounds belonging to the general class of substituted pentanedioic acids, where the substituted moiety might be (subclass 1) a sulfanyl alkyl group; (subclass 2) an halogenated benzyl and a phosphinyl group; (subclass 3) a phosphonomethyl group, for prevention, management and/or treatment of hyperaggressive behavior arising from environmental or social conditions, injury or disease. Compounds including subclasses 1,2 & 3 as well as [2-(pentafluorophenylmethyl) hydroxyphosphinyl]methyl-pentanedioic acid, and/or other related substituted pentanedioic acids that inhibit NAALADase or mimic NAAG, and/or 2-(phosphonomethyl)-pentanedioic acid (2-PMPA) and/or beta-N-acetyl-aspartyl-glutamate (NAAG) and/or alpha N-acetyl-aspartyl-glutamate are appropriate means for prevention, management and/or treatment of hyperaggressive behavior arising from environmental or social conditions, injury or disease.

### BACKGROUND OF THE INVENTION

It is known that the neurotransmitter, N-Acetyl-aspartyl-glutamate (NAAG), the most abundant neuropeptide in the central nervous system, can be released from neurons in response to depolarization. NAAG then can be enzymatically converted to the excitatory neurotransmitter, glutamate (Glu), by the enzyme alpha-linked acidic dipeptidase (NAALADase), also known as NAAG-hydrolyzing enzyme or glutamate carboxy pepetidase II (GCP II [E.C. number 3.4.17.21]). This important reaction occurs under both normal and pathophysiological conditions of the nervous system.

Because hydrolysis of NAAG by liberates the more potent excitatory amino acid neurotransmitter, Glu, NAAG has been regarded as a storage form of synaptic Glu. NAALADase has been proposed to modulate downstream neuronal excitability by regulating the synaptic availability of Glu. Thus an inhibitor of NAALADase activity would diminish glutamatergic tone by three convergent mechanisms: directly inhibiting liberation of Glu from the precursor NAAG, resulting in increased levels of NAAG. NAAG itself can diminish overall glutamatergic tone via two mechanisms: (1) NAAG can inhibit GLU release through activation of presynaptic mGluR3 receptors; (2) because NAAG has only a fraction of the excitatory potency of glutamic acid at the NMDA receptor, it can act as an glutamate antagonist at that receptor.

Inhibiting enzymatic conversion of NAAG to glutamate using compounds of subclasses 1,2 or 3, or beta-NAAG or 2-PMPA is a useful approach because the convergent triple mechanism may require less drug than would an approach aimed solely at blocking the NMDA receptor. Drugs blocking the NMDA receptor have been associated with unacceptable behavioral side effects.

### SUMMARY OF THE INVENTION

The instant invention is related to the use of compositions containing compounds of subclasses 1,2 or 3 to include [2-[(pentafluorophenylmethyl) hydroxyphosphinyl]methyl-pentanedioic acid, and/or other related substituted pentanedioic acids that inhibit NAALADase or mimic NAAG, and/or 2-(phosphonomethyl)-pentanedioic acid (2-PMPA) and/or beta-N-acetyl-aspartyl-glutamate (beta-NAAG, a non-hydrolyzable analog of the naturally-occuring alpha-NAAG) and/or alpha N-acetyl-aspartyl-glutamate as appropriate means for prevention, management and/or treatment of hyperaggressive behavior arising from environmental or social conditions, injury or disease.

### DETAILED DESCRIPTION OF THE INVENTION

It is well-established that housing in isolation for significant periods of time will enhance the natural tendency of cage resident mice to attack an “intruder” mouse placed in their home cage. The isolation model in mice is the most appropriate model for screening drugs for anti-aggression potential. We tested whether acute exposure to the NAALADase inhibitor [2-[(pentafluorophenylmethyl) hydroxyphosphinyl]methyl-pentanedioic acid, administered prior to a social interaction test, would inhibit aggressive behavior in SJL mice that had been individually housed for eight months. [2-[(pentafluorophenylmethyl) hydroxyphosphinyl]methyl-pentanedioic acid (30 mg/kg, i.p.) was administered 30 minutes prior to a social interaction test to pharmacologically naive male mice which had been selected and behaviorally shaped to be hyperaggressive, and had consistently shown markedly aggressive behaviors in prior interactions with intruder mice. Administration of [2-[(pentafluorophenylmethyl) hydroxyphosphinyl]methyl-pentanedioic acid (30 mg/kg, i.p.) inhibited aggression, indicated by greater latencies to display tail-rattling, attack and bite, and fewer tail-rattling responses to a non-aggressive conspecific, relative to vehicle. In addition, fewer mice that received [2-[(pentafluorophenylmethyl) hydroxyphosphinyl]methyl-pentanedioic acid (30 mg/kg) initiated attack, relative to those that received vehicle.

Long-term individual housing in mice increases aggression, indicated by shorter latency to attack conspecifics; this phenomenon has been termed isolation-induced aggression (Yen et al., 1959 as seen in Miczek and O=Donnell, 1978; Anton et al., 1968; Brain and Nowell, 1971; reviewed in Miczek, 1987). Acute administration of antidepressants (reviewed in File, 1986), and anxiolytics including 5-HT1A agonists and benzodiazepines, reduce isolation-induced aggression (White et al., 1991; Skolnick et al., 1985). NMDA antagonists reduce both isolation-induced aggression (Belozertseva and Bespalov, 1999) and opioid withdrawal induced aggression (Sukhotina and Bespalov, 2000). Since many NMDA antagonists, including ketamine, PCP and MK-801 have psychomimetic effects (see Wedzony et al., 2000), there is interest in developing novel glutamatergic compounds that have therapeutic potential but fewer adverse side effects.

We hypothesized that similar to the effects of NMDA antagonists, NAALADase inhibition and therefore increased NAAG, would inhibit aggressiveness under conditions of high basal levels of aggressiveness. We administered the NAALADase inhibitor [2-[(pentafluorophenylmethyl) hydroxyphosphinyl]methyl-pentanedioic acid prior to a social interaction test in a novel cage and measured behavioral responses to a non-aggressive conspecific; a novel cage was used to lower the levels of aggressiveness. The paradigm used was adapted from File (reviewed in Crawley, 2000).

Principles of laboratory animal care (NIH publication No. 85-23, revised 1985) were followed. Male SJL subject mice (30-35 g; Jackson Laboratories) were housed in reverse 12:12 light-dark cycle (lights off 0900) in a temperature (20-23° C.) and humidity (50-20%) controlled room, with food and water available ad libitum and were housed individually in large cages (48×27×20 cm) for 8 months, during which time subjects were exposed weekly to non-aggressive C57BL/6 mice that had received olfactory bulbectomies (obx), since these mice do not attack (Denenberg et al., 1973). In addition, these mice had been used to socially defeat mice that were used as subjects in numerous other studies. These highly aggressive SJL had no previous drug exposure. At the end of 8 months of training, subjects weighed 30-37 grams and had attack latencies of less than 30 sec when exposed to an obx. During the social interaction test, two C57BL/6 male mice (30-33 g; Jackson Laboratories), which were screened for non-aggressiveness and unfamiliar to the subjects, were used as stimulus mice.

Procedure:

[2-[(pentafluorophenylmethyl) hydroxyphosphinyl]methyl-pentanedioic acid, MW 390.2 (3 mg/kg or 30 mg/kg in 50 mM Hepes in 0.9% saline; pH 7.2; Guilford Pharmaceuticals) or vehicle (50 mM Hepes in 0.9% saline; pH 7.2) was administered intraperitoneally (i.p.; 10 ml/kg) 30 min. prior to a 4 min. social interaction test. Subject mice were placed in a novel cage (48×27×20 cm) along with a non-aggressive male conspecific and tested under low illumination (red lights) during the dark phase of their cycle. Methods for various social interaction tests reviewed in Crawley et al. (2000). Tests were videotaped and behaviors were scored using a computer program (Hindsight, Scott Weiss, UK), by two observers blind to the treatment. Behavioral measures included 1) measures of aggressiveness or threat: attack, bite, chase or follow, grooming conspecific, tail rattling 2) measures of exploratory and locomotor activity: walking, rearing, digging, 3) measures of defensiveness: defensive posture (crouch, upright) and flight. Details on behavioral measurements may be found in Grant and McIntosh, 1963; Lumley et al., 2000; Crawley, 2000.

Statistics:

One-way analysis of variance was performed for each behavioral measure with drug dose as the independent factor. Data were further probed using Dunnet=s test. If a behavioral response was not displayed, a maximum latency of 240 seconds was scored.

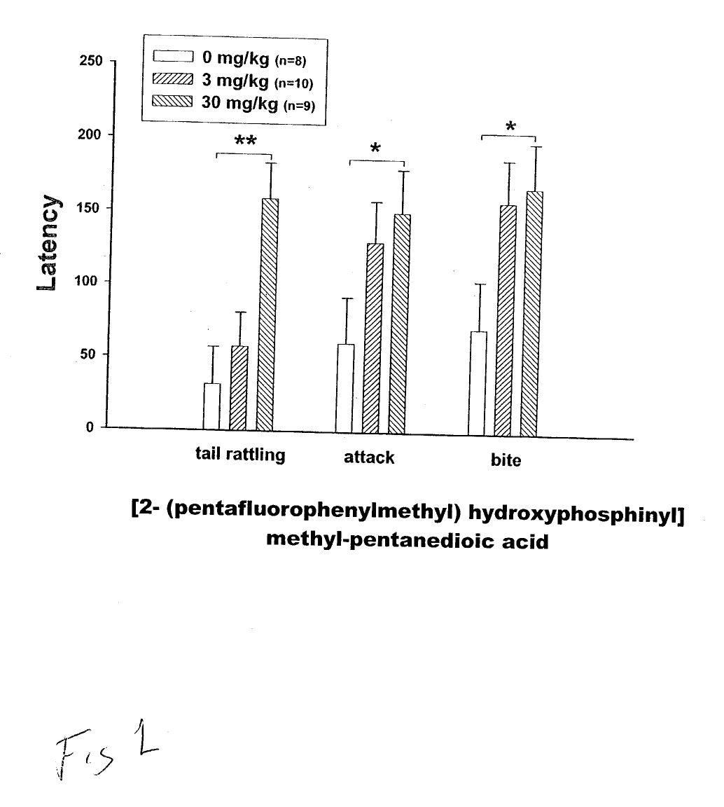

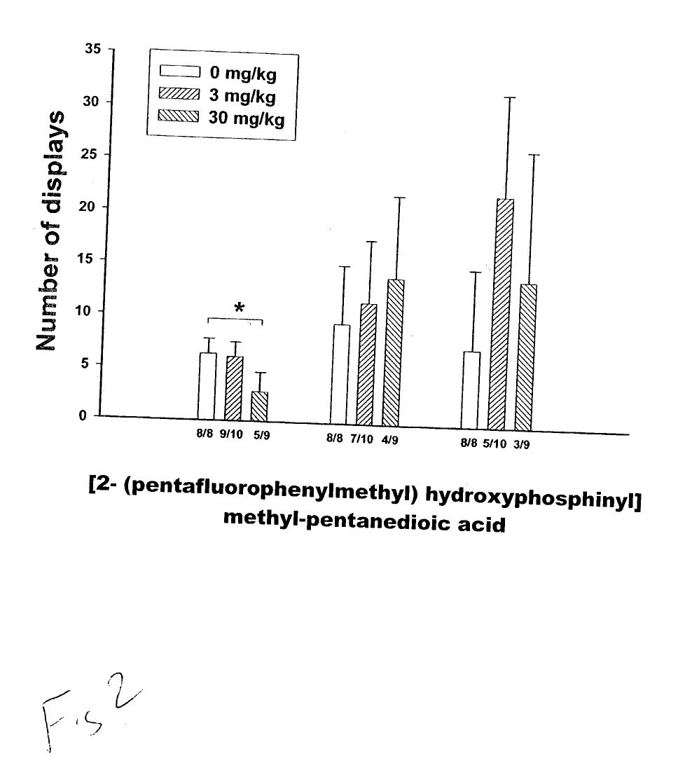

Results:

[2-[(pentafluorophenylmethyl) hydroxyphosphinyl]methyl-pentanedioic acid (30 mg/kg, i.p.) inhibited aggressiveness in long term individually housed mice, as indicated by increased latencies to display tail-rattling (F (2, 26)=7.41), attack (F (2, 26)=2.36) and bite (F (2, 26)=2.82) in GPI-treated mice relative to vehicle-treated mice (FIG. 1). Mice that received [2-[(pentafluorophenylmethyl) hydroxyphosphinyl]methyl-pentanedioic acid (30 mg/kg) tended to display fewer tail-rattling responses, relative to vehicle-treated mice (F (2, 26)=3.66; FIG. 2).

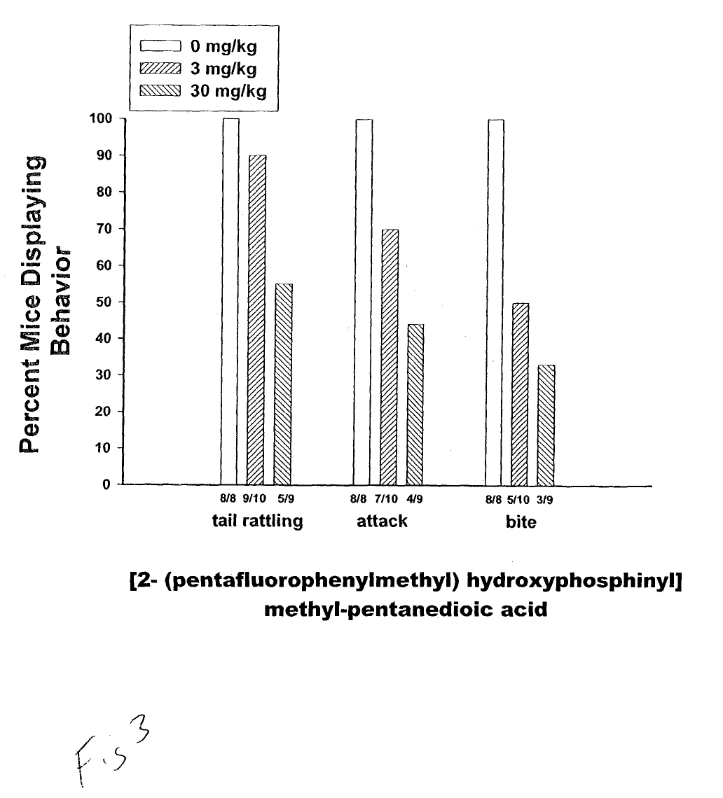

There was no effect of [2-[(pentafluorophenylmethyl) hydroxyphosphinyl]methyl-pentanedioic acid on the number of attacks (F (2,18)=0.11) or bites (F (2, 26)=0.31; FIG. 2) in mice displaying these behaviors. Only 4 of 9 mice that received 30 mg/kg [2-[(pentafluorophenylmethyl) hydroxyphosphinyl]methyl-pentanedioic acid attacked and 7 of 10 mice that received 3 mg/kg [2-[(pentafluorophenylmethyl) hydroxyphosphinyl]methyl-pentanedioic acid, while all 8 of 8 vehicle-treated mice attacked (see FIG. 3). Few mice displayed any defensive responses; there was no effect of [2-[(pentafluorophenylmethyl) hydroxyphosphinyl]methyl-pentanedioic acid on defensive responses.

Administration of the NAALADase inhibitor [2-[(pentafluorophenylmethyl) hydroxyphosphinyl]methyl-pentanedioic acid, which increases level of the peptide neurotransmitter NAAG, inhibited aggressiveness in highly aggressive mice that had been individually housed long-term. Mice that received the higher dose of [2-[(pentafluorophenylmethyl) hydroxyphosphinyl]methyl-pentanedioic acid displayed greater latencies to display tail-rattling, considered a component of aggressive behavior (Miczek, 1978), and greater latencies to attack and bite the conspecific, relative to vehicle-treated mice. In addition, mice treated with [2-[(pentafluorophenylmethyl) hydroxyphosphinyl]methyl-pentanedioic acid had more tail rattling responses than vehicle. These effects of inhibited aggression might have been mediated by its effect as a partial agonist on NMDA receptors. Acute administration of a variety of NMDA antagonists inhibited aggressiveness in Swiss mice with isolation-induced aggressiveness (Belozertseva and Bespalov, 1999) and in SHR mice with morphine withdrawal-induced aggressiveness (Sukhotina and Bespalov, 2000). In contrast, others reported that the NMDA antagonist phencyclidine (PCP) administered to individually housed mice increased aggressiveness (Wilmot et al., 1987). However, PCP reduced the number of mice that displayed attack, in agreement with the current findings that only 44% of mice that received 30 mg/kg GPI 5232 attacked the conspecific, relative to 100% of mice that received vehicle. In the current study, the 4 of 9 mice treated with [2-[(pentafluorophenylmethyl) hydroxyphosphinyl]methyl-pentanedioic acid that attacked tended to have more attacks than the vehicle-treated mice, although this effect was not significant. The effect of NAALADase inhibition on aggression may be mediated by decreased glutamate, either through inhibition of glutamate synthesis from NAAG or through NAAG activation of mGluR presynaptic receptors, which inhibits glutamate release. Anxiolytic effects of an mGluRII agonist, and mGluRIII and mGluRIV antagonists have been reported (Monnet al., 1997; Helton et al., 1998; Chojackna et al., 1996; Spooren, 2000). Whether these ligands also affect aggression is unclear.

The basal level of aggressiveness is probably a key factor in the effects of both NMDA antagonists and NAALADase inhibition. There have been reports that NMDA antagonists increase aggressiveness when basal levels are low. In mice, the NMDA antagonist dizocilpine increased aggression (McAllister, 1990). Krsiak (1984) suggested that PCP might increase aggressiveness in mice with low baseline levels of aggressiveness, similar to previously observed effects of chronic exposure (i.c.v.) of C57BL/6 mice to the NAALADase inihibitor 2-PMPA (Lumley, Soc. Neurosci). In morphine-naïve SHR mice (low aggressiveness), acute administration of NMDA antagonists did not affect aggressive behavior (Sukhotina and Bespalov, 2000). Also, when basal levels of aggressiveness are low, NMDA antagonists increased social interaction, decreased anxiety, inhibited fear-conditioning, and increased aggressiveness (Jessa et al., 1995; Chojnacka-Wojcik et al., 1997; Ossawska et al., 1997). Studies using isolation to induce aggressiveness typically individually house subjects for approximately 30 days (reviewed in Crawley, 2000). In the current study, basal levels of aggressiveness were extremely high since subjects were individually housed for 8 months and paired briefly with subordinate mice once/week. Whether [2-[(pentafluorophenylmethyl) hydroxyphosphinyl]methyl-pentanedioic acid inhibits aggression in mice individually housed for a shorter time period remains to be determined.

Long-term individual housing induces a variety of neurochemical changes, including effects on amino acids. Individual housing of mice in small cage for over 2 months, increased levels of glutamate in the colliculi and cortex (Cordoba et al., 1987). Other neurochemical changes induced by long-term isolation include decreased serotonin_{1A} receptor number and affinity, and decreased serotonin turnover in mice (Popova and Petkov, 1990; Valzelli and Bernasconoi, 1979), and increased catecholamine turnover in rats (Miachon et al., 1993). Long term individual housing induces hormonal effects as well, including decreased glucocorticoids in rats and mice (Brain 1971; Miachon et al., 1993), increased prostate weights suggesting increased gonadotropins (Brain 1970; 1971), and decreased thyrotropin releasing hormone in mice (Mainardi et al, 1984). In addition, isolation increased hypothalamic nerve growth factor in mice (Spillantini, 1989). Whether and how these neurochemical changes interact with the effect of NAALADase inhibition on aggressive behavior is unclear.

Aggressive behavior arises in certain patients suffering from a large and diverse group of neurological and psychiatric disorders. The neurological or neurodegenerative diseases include but are not limited to: Alzheimer's Disease, Creutzfeld Jacob disease and Bovine Spongiform Encephalitis (“Mad Cow Disease”), other forms of encephalitis or infection in the central nervous system, chronic wasting disease, other dementias (e.g. Dementia Pugilistica, Parkinsons, Pick's Disease, Huntington's Disease, and HIV AIDS), brain injury, organic brain syndrome, tertiary Syphilis, Korsakoff's psychosis, brain tumors, cerebral ischemia, seizure disorders (convulsive and non-convulsive). Psychiatric diseases of interest in this regard include but are not limited to, developmental disorder or personality disorder, mental retardation, autism, bipolar disorder, mood disorders, psychoactive substance intoxication and/or withdrawal, psychotic disorders, premenstrual dysphoric disorder, posttraumatic stress disorder, panic disorder, generalized anxiety disorder, conduct disorder, adjustment disorder, antisocial personality disorder, borderline personality disorder, intermittent explosive disorder, attention deficit/hyperactivity disorder, major depressive disorder and dysthymia.

Aggression itself is a behavior, not a disease. Specific treatments for a given disease may not prevent or control the development of aggressive behavior. Since uncontrolled aggressive behavior is not acceptable, its management is a major problem in health care facilities, and the long term use of restraints is not considered humane, there is a need for a pharmacological intervention should aggressive behavior begin to appear in a patient. The medications currently available have many undesirable side effects, including such behavioral toxicities as, ataxia, dystonia, orthostatic hypotension, oversedation. Benzodiazepines have been associated with paradoxical excitement, as well as with development of dependence. Accordingly the instant invention is envisioned as an advance in patient management that could be complementary to the primary pharmacotherapy for the neurologic or psychiatric disorder.

A number of patents (including, but not limited to U.S. Pat. No. 6,228,888; U.S. Pat. No. 6,458,775; U.S. Pat. No. 6,413,948) have taught the use of the compounds of subclasses 1,2 & 3 for the treatment of anxiety or dementia arising in the course of the aforementioned neurological and psychiatric diseases. These patents (which are incorporated herein by reference in their entirety) teach the use of compounds disclosed herein for anxiety and for dementia, but there is no suggestion therein that these compounds may be used for prevention or treatment of pathological aggression or hyperaggressive behavior.

### ROUTE OF ADMINISTRATION

In the methods of the present invention, the compounds may be administered parenterally, orally, by inhalation spray, rectally, nasally, buccally, vaginally, topically, or via an implanted reservoir in dosage formulations containing conventional non-toxic pharmaceutically-acceptable carriers, vehicles and/or adjuvants. The term parenteral as used herein includes subcutaneous, intravenous, and intramuscular.

To be effective therapeutically via central nervous system actions, the NAALADase inhibitors used in the methods of the present invention should readily penetrate the blood-brain barrier when peripherally administered. Compounds that penetrate poorly can be effectively administered intravenously, if contained in liposomes.

The compounds may also be administered in the form of sterile injectable preparations, for example, as sterile injectable aqueous or oil-based suspensions. These suspensions can be formulated according to techniques known in the art using suitable dispersing or wetting agents and suspending agents. The sterile injectable preparations may also be sterile injectable solutions or suspensions in non-toxic parenterally acceptable diluents or solvents, for example, as solutions in 1,3-butanediol. Among the acceptable vehicles and solvents that may be employed are water, Ringer's solution and isotonic sodium chloride solution. In addition, sterile oils are conventionally employed as solvents or suspending mediums. For this purpose, an oil such as a synthetic mono- or, di-glyceride may be employed. Fatty acids such as oleic acid and its glyceride derivatives, including olive oil and castor oil, especially in their polyoxyethylated forms, are useful in the preparation of injectables. These oil solutions or suspensions may also contain long-chain alcohol diluents or dispersants.

Additionally, the compounds may be administered orally in the form of capsules, tablets, aqueous suspensions or solutions. Tablets may contain carriers such as lactose and cornstarch, and/or lubricating agents such as magnesium stearate. Capsules may contain diluents including lactose and dried cornstarch. Aqueous suspensions may contain emulsifying and suspending agents combined with the active ingredient. The oral dosage forms may further contain sweetening and/or flavoring and/or coloring agents.

The compounds may further be administered rectally in the form of suppositories. These compositions can be prepared by mixing the drug with suitable non-irritating excipients that are solid at room temperature, but liquid at rectal temperature such that they will melt in the rectum to release the drug. Such excipients include cocoa butter, beeswax and polyethylene glycols.

The NAALADase inhibitors used in the methods of the present invention may be administered by a single dose, multiple discrete doses or continuous infusion. Since the compounds are small, easily diffusible and relatively stable, they are well suited to continuous infusion. Pump means, particularly subcutaneous pump means, are preferred for continuous infusion.

### DOSAGE

Dose levels on the order of about 0.1 mg to about 10,000 mg of the active ingredient compound are useful in the treatment of the above conditions, with preferred levels being about 0.1 mg to about 1,000 mg. The specific dose level for any particular patient will vary depend upon a variety of factors, including the activity of the specific compound employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful. The considerations for determining the proper dose levels are well known in the art.

In a preferred embodiment, the NAALADase inhibitors are administered in lyophilized form. In this case, 1 to 100 mg of a NAALADase inhibitor may be lyophilized in individual vials, together with a carrier and a buffer, such as mannitol and sodium phosphate. The compound may be reconstituted in the vials with bacteriostatic water before administration.

In treating aggression, the NAALADase inhibitors are preferably administered orally or parenterally at least 1 to 6 times daily, and may follow an initial bolus dose of higher concentration.

As previously mentioned, the NAALADase inhibitors used in the methods of the present invention may be administered in combination with one or more therapeutic agents. Specific dose levels for these agents will depend upon considerations such as those identified above for the NAALADase inhibitors.

### ADMINISTRATION REGIMEN

For the methods of the present invention, any administration regimen regulating the timing and sequence of drug delivery can be used and repeated as necessary to effect treatment. Such regimen may include pretreatment and/or co-administration with additional therapeutic agents.

To maximize protection of nervous tissue from nervous insult, the NAALADase inhibitors should be administered to the affected cells as soon as possible.

### COMBINATION WITH OTHER TREATMENTS

The NAALADase inhibitors can be co-administered with one or more therapeutic agents either (a) together in a single formulation, or (b) separately in individual formulations designed for optimal release rates of their respective active agent. Each formulation may contain from about 0.01% to about 99.99% by weight, preferably from about 3.5% to about 60% by weight, of a NAALADase inhibitor, as well as one or more pharmaceutical excipients, such as wetting, emulsifying and pH buffering agents.

Dosage of compound selected from subclasses 1,2 or 3, as well as of beta-NAAG and PMPA are essentially the same, and will, of course, depend on the age, size and condition of the patient or mammal as well as on the mode of administration. Dosage in mg/kg may be reduced as size and weight increase.

For purposes of obtaining beneficial effects on the CNS, the agents of the invention can be delivered by any means that allows the active agent to reach regions critical for mediating the behavior of interest. Carriers that are appropriate are those that are basically non-irritating such as buffered saline that may contain glucose and other active agents such as antibiotics and anti-inflammatory agents. The concentration of PMPA is usually 0.05% to 6%. For example, a solution containing 0.5% PMPA in 5% glucose in phosphate buffered saline may be administered intravenously.

The agents of the invention can be delivered by any means that allows the active agent to reach target areas of the CNS. Carriers such as polyvinyl alcohol are appropriate, as are those that are basically non-irritating such as buffered saline containing 5% polyvinyl alcohol of such weight (number) as to be soluble in water.

Without relying on any particular theory for novelty, it is believed that these beneficial effects may result from competition for the glutamate receptor by alpha- and beta-NAAG acting as a partial agonist at the N-methyl,D-aspartate (NMDA) receptor complex. It is also possible that the treatment results in increased effects of alpha-NAAG as an agonist at the presynaptic mGluR3 receptor, where it inhibits direct release of Glu from those neurons.

The active agents of the invention are either readily available commercially or can readily be synthesized. Compositions containing PMPA and alpha- and beta-NAAG may be administered for treatment by injection. Appropriate carriers include saline, glucose (5% or 10% being more usual) in half normal saline, and buffered saline, etc. Compositions may also contain other agents used as carriers such as dimethylsulfoxide (DMSO).

The dosage administered will depend on the condition, size and age of the patient. In general, dosage of 0.0001/Kg to 1 mg/Kg for PMPA, alpha-NAAG or beta-NAAG will be appropriate, with larger animals such as man receiving about 0.0001 to 0.05 mg/Kg and smaller animals receiving larger dosage relative to weight.

Compositions for use in the method of this invention may contain, in addition to PMPA, other active agents such as antipsychotics, anxiolytics, antidepressants, neurotrophins, antioxidants, anti-inflammatory agents, antibiotics, anesthetics and analgesics.

Chronic depot delivery systems known in the art may be used to deliver the active agents.

### EXAMPLE 1

A composition for administration by injection is prepared by adding 3 mg PMPA to 3 ml DMSO. To this is added sufficient 5% glucose in half-normal saline to make 100 ml solution.

### EXAMPLE 2

A composition is prepared for use as injection. Six mg of beta-NAAG is added to 3 ml DMSO. To this is then added sufficient saline to make 100 ml of solution.

### EXAMPLE 3

A composition is prepared for injection by adding sufficient 10% glucose in half-normal saline to 3 mg of alpha-NAAG to provide 100 ml of solution.

It is urged that the above examples are provided for purposes of general instruction and are not to be construed as in any way limiting the scope of the invention.

What we claim is:

1. A method of treating or protecting against pathological aggression in a subject in need thereof comprising administration of a composition of matter comprising a glutamate-blocking, glutamate release-inhibiting and/or glutamate-formation-inhibiting effective amount of an active agent chosen from compounds belonging to the general class of substituted pentanedioic acids, where the substituted moiety might be (subclass 1) a sulfanyl alkyl group; (subclass 2) a halogenated benzyl and a phosphinyl group; (subclass 3) a phosphonomethyl group; and/or 2-(phosphonomethyl)-pentanedioic acid (2-PMPA) and/or beta-N-acetyl-aspartyl-glutamate (NAAG) 1-(phosphonomethyl) pentanedioic acid, alpha N-acetyl-aspartyl-glutamate, and beta N-acetyl-aspartyl-glutamate in a pharmaceutically acceptable carrier.

2. A method of claim 1 wherein the hyperaggressive behavior or pathological aggression arises from drug-induced effects, intoxication dependency or withdrawal.

3. A method of claim 1 wherein the hyperaggressive behavior or pathological aggression arises from neurological or neurodegenerative disease or dementia, including but not limited to: Alzheimer's dementia, Creutzfeld Jacob disease and Bovine Spongiform Encephalitis (“Mad Cow Disease”), other forms of encephalitis or infection in the central nervous system, chronic wasting disease, other dementias (e.g. Dementia Pugilistica, Parkinsons, Pick's Disease, Huntington's Disease, and HIV AIDS), brain injury, organic brain syndrome, Korsakoff's psychosis, brain tumors, cerebral ischemia, seizure disorders (convulsive and non-convulsive).

4. A method of claim 1 wherein the hyperaggressive behavior or pathological aggression arises from psychiatric disease, developmental disorder or personality disorder, including but not limited to mental retardation, autism, bipolar disorder, mood disorders, psychoactive substance intoxication and/or withdrawal, psychotic disorders, premenstrual dysphoric disorder, posttraumatic stress disorder, panic disorder, generalized anxiety disorder, conduct disorder, adjustment disorder, antisocial personality disorder, borderline personality disorder, intermittent explosive disorder, attention deficit/hyperactivity disorder, major depressive disorder and dysthymia.

5. A method of claim 1 wherein composition contains 0.5% to 6% of compounds of subclasses 1,2 or 3, 2-PMPA, alpha-NAAG or beta-NAAG.
